(19) United States
(12) Patent Application Publication
(10) Pub. No.: US 2024/0165664 A1
(43) Pub. Date: May 23, 2024

(54) METHOD FOR DRIVING A WAVE EMITTER AND RECEIVER DEVICE

(71) Applicant: SUPERSONIC IMAGINE, Aix-En-Provence (FR)

(72) Inventors: Hubert CORDONNIER, Aix en Provence (FR); Guillaume ROUX, Aix en Provence (FR); Jean-Alain MARTIN, Aix en Provence (FR)

(21) Appl. No.: 18/503,115

(22) Filed: Nov. 6, 2023

(30) Foreign Application Priority Data
Nov. 18, 2022 (FR) FR2212005

(51) Int. Cl. B06B 1/02; A61B 8/00

(52) U.S. Cl. CPC B06B 1/0215; A61B 8/54; B06B 2201/55; B06B 2201/76

(57) ABSTRACT

The present disclosure relates to a method and a system for driving a first wave emitter and receiver device. The method includes driving the first device throughout channels established via electrical links, wherein at least two of said channels are permuted over time among the electrical links.

### PRIOR ART

A wave emitter and/or receiver device, such as a transducer device, usually comprises a plurality of transducer elements (for example arranged in an array) for communication, imaging or scanning purposes, for example in the medical imaging, radar, sonar, seismology, wireless communications, radio astronomy, acoustics and biomedicine fields. An example comprises ultrasound imaging.

To this end, the wave emitter and/or receiver device may be driven by means of electrical signals, these signals being for example transmitted between the wave emitter and/or receiver device and a control unit. Thus, electrical signals representing waves can be transmitted to transducer elements of the wave emitter and/or receiver device, causing the emission of waves in a given medium. Electrical signals may possibly be recovered in return, these signals representing a response (or echo) of the medium to the undulatory excitation.

For example, the aim of the ultrasound imaging may be to estimate the reflectivity of a medium. The predefined frequency may then be selected depending on the characteristics of the medium (for example human tissue).

More particularly, in a conventional ultrasound imaging process, it is possible for example to use an ultrasound transducer device (also called ultrasound probe) equipped with at least one or one set of ultrasound transducer elements. In such a process, one or more transducer(s) are used to convert the electrical signals into ultrasonic waves. In particular, the transducers may emit one or successively several ultrasonic beams in the direction of a medium, which corresponds to an emission operation. Afterwards, in a reception operation, a set of backscattered echo signals are received from the medium by the same set or by another set of transducer elements. In particular, each of the transducer elements may, for example, convert a received echo signal into an electrical signal. Afterwards, the signal can be processed by the ultrasound system or by any associated (dedicated) system, directly connected or not. For example, the signal may be amplified, filtered, digitised and/or a signal conditioning operation may be performed. The transducer elements may be disposed according to a row of transducers, an array, or as a network of transducers or any other configuration.

However, driving wave emitter and/or receiver device, such as transducer devices for example, does not always allow obtaining electrical signals in a satisfactory manner. It has been noticed that the electrical signals transmitted to, or received from, the wave emitter and/or receiver devices are sometimes disturbed or are not optimum, which could lead to limited or inadequate performances of the system. In particular, these disturbances could for example disturb driving of a wave emitter and/or receiver device and/or result in the reception of degraded feedback data, which could then lead to a degraded image quality in imaging applications (for example ultrasound imaging).

### DESCRIPTION OF THE DISCLOSURE

One of the objects of the present disclosure is to solve at least one of the previously described problems or deficiencies.

In particular, it might be desirable to improve driving of a wave emitter and/or receiver device, in particular, yet not exclusively, of an ultrasound transducer device, and/or to improve the quality of the signals of such a wave emitter and/or receiver device.

In particular, as described in more detail later on, electrical noise (which could comprise in particular electronic noise) might disturb the signals transmitted in electrical links used to drive a wave emitter and/or receiver device. Thus, it might be desirable to limit the effects induced by such an electrical noise, in particular in order to limit the disturbances that could affect the electrical signals transmitted between a control system and a wave emitter and/or receiver device.

For example, in the case where the emitter and/or receiver device is (or comprises) an ultrasound probe, it might be desirable to improve the electrical signals transmitted throughout electrical links by limiting the electrical noise that might disturb this transmission, for example in order to improve the quality of an ultrasonic image.

To this end, according to a first aspect, the present disclosure relates to a method for driving a wave emitter and/or receiver device. The method according to the first aspect comprises driving the first device throughout channels established via electrical links, wherein at least two of said channels are permuted over time among the electrical links.

By providing such a method, it becomes advantageously possible to improve driving of the wave emitter and/or receiver device and therefore the performances of the system as a whole, in particular by smoothing, or averaging, or redistributing (over time and/or space), the electrical noise that might be produced among the electrical links used to transmit the electrical signals, so as to limit the possible disturbances that might result from such an electrical noise. The quality of the images built based on the emitted signals and those received in return is thus substantially improved for example, in the context of an ultrasound imaging application.

The method may be implemented with a minimum adaptation in existing drive systems, by implementing the means necessary to carry out the channel permutation(s) in a software and/or hardware form. The costs for implementing the method may therefore be reduced.

Thus, for example, it is sometimes no longer necessary to implement a correction algorithm to limit the defects occurring on the image because of the electrical noise, which allows facilitating the processing of the signals and limiting the associated processing costs in particular in terms of time and resources.

The method according to the disclosure may include other features that may be taken separately or in combination, particularly from the following embodiments.

According to an embodiment, the driving comprises:
establishing the channels by a drive device.

According to one embodiment, the driving further comprises:
transmitting electrical signals throughout the channels.

According to an embodiment, said channels are analog channels for the transmission and/or reception of electrical signals between the control device and the first device.

According to an embodiment, the method comprises at least one permutation over time of said at least two channels among the electrical links.

According to an embodiment, the permutation comprises a plurality of permutations over time of the channels among the electrical links.

According to an embodiment, the channels are permuted during the permutation so that one single electrical link is used at each time per channel.

According to an embodiment, the permutation is carried out according to a predetermined sequence over time.

According to an embodiment, the permutation is carried out according to a random sequence over time.

According to an embodiment, the electrical links are interposed between a first multiplexer and a second multiplexer, each permutation being caused by a change in routing by the first and second multiplexers of the channels among the electrical links.

According to an embodiment, the first and second multiplexers carry out a multiplexing of the channels between a plurality of pulsers and a plurality of piezoelectric elements, each channel coupling the same pulser to the same piezoelectric element independently of the permutation.

According to an embodiment, the electrical links comprise at least one intermediate multiplexer dividing said electrical links into sections mounted in series, wherein the method comprises at least one said permutation which permutes the channels among the sections of the electrical links.

According to an embodiment, the driving comprises:
sending groups of electrical signals to the first device throughout the channels, each group corresponding to a shot configured to cause the emission of a wave by the first device;
wherein said at least two channels are permuted every X shots among the electrical links, X being an integer greater than or equal to 1 (and possibly greater than or equal to 2).

According to an embodiment, said waves are acoustic waves.

According to an embodiment, said method is applied to ultrasound medical imaging.

According to a second aspect, the present disclosure relates to a computer program comprising instructions which, when the program is executed by a computer, cause the latter to implement the method according to the first aspect. In other words, the different steps of the method according to the first aspect are determined by instructions of computer programs.

Such a computer program may use any programming language or the like, and it may be in the form of a source code, an object code, or an intermediate code between a source code and an object code, such as in a partially compiled form, or in any other desirable form.

According to a third aspect, the present invention relates to a recording medium (or information medium), readable by a computer (or a processor), on which a computer program is recorded comprising instructions for the execution of the steps of a method according to the first aspect of the present disclosure.

On the one hand, the recording medium may consist of any entity or device capable of storing the program, such as at least one volatile and/or non-volatile memory. For example, the medium may include a storage means, such as a rewritable non-volatile memory, a ROM memory, a CD-ROM or a ROM memory of the microelectronic circuit type, or a magnetic recording means or a hard disk. For example, this memory may comprise a graphic card (or video card) memory, this memory type being in particular able to process image data (or video data).

On the other hand, this recording medium may also be a transmissible medium such as an electrical or optical signal, such a signal being able to be conveyed via an electrical or optical cable, by conventional or hertzian radio or by self-directed laser beam or by other means. In particular, the computer program according to the present disclosure may be downloaded thanks to a wired or wireless network, of the local type (Bluetooth® for example, Wi-Fi, Ethernet, Internet, 4G, 5G inter alia).

Alternatively, the recording medium may be an integrated circuit in which the computer program is incorporated, the integrated circuit being adapted to execute or to be used in the execution of the considered method.

According to a fourth aspect, the present disclosure relates to a control system for driving a first wave emitter and/or receiver device, said system comprising:
a control device configured to drive the first device throughout channels established via electrical links; and
a drive device configured to permute at least two of said channels over time among the electrical links.

According to an example, the control system comprises a memory associated with a processor configured for the implementation of the steps of the method according to the first aspect of the present disclosure.

The control system may have functions that correspond to the operations of the method according to the first aspect of the disclosure. In particular, the different embodiments mentioned hereinbefore in connection with the method according to the first aspect of the disclosure as well as the associated advantages may apply similarly to the control system according to the fourth aspect of the invention.

The features and advantages of the disclosure will appear more clearly upon reading the following description, given only as a non-limiting example, and made with reference to the appended figures. In particular, the examples illustrated in the figures may be combined together, except in case of obvious incompatibility.

### BRIEF DESCRIPTION OF THE FIGURES

Other features and advantages of the present disclosure will appear from the description of the non-limiting embodiments of the present disclosure hereinafter, with reference to the appended FIGS. 1 to 12, wherein:

FIG. 1 schematically illustrated the consequences, in an example of an ultrasonic image, of the electrical noise disturbing the electrical signals conveyed throughout electrical links, according to a particular example;

FIG. 2 shows a schematic drawing of a system for driving a wave emitter and/or receiver device, according to embodiments of the present disclosure;

FIG. 3 shows a schematic drawing of an ultrasound imaging system according to embodiments of the present disclosure;

FIG. 4 shows a schematic drawing of a system for driving an ultrasound transducer device according to embodiments of the present disclosure;

FIG. 5 schematically illustrates the channel permutation in the system of FIG. 4, according to embodiments of the present disclosure;

FIG. 6 schematically illustrates the electrical signals transmitted in the channels of the system of FIG. 4 according to embodiments of the present disclosure;

FIG. 7 shows a schematic drawing of a system for driving an ultrasound transducer device according to embodiments of the present disclosure;

FIG. 8 is a diagram schematically illustrating a drive method according to embodiments of the present disclosure;

FIG. 9 is a time chart schematically illustrating channel permutations according to embodiments of the present disclosure;

FIG. 10 is a time chart schematically illustrating channel permutations according to embodiments of the present disclosure;

FIG. 11 is a time chart schematically illustrating channel permutations according to embodiments of the present disclosure;

FIG. 12 shows a schematic drawing of a system for driving an ultrasound transducer device, according to embodiments of the present disclosure.

### Description of the Embodiments

As already indicated, the present disclosure covers driving wave emitter and receiver devices and methods for driving such devices. Such a device may consist of a medical device. More particularly, it may belong to an ultrasonography system.

A wave emitter and/or receiver device may be driven by means of electrical signals, these signals being for example transmitted between the wave emitter and/or receiver device and a control unit of a control system. These electrical signals may be transmitted or exchanged between the control unit and the wave emitter and/or receiver device (such as a transducer device for example) throughout electrical links. For example, each piezoelectric element (or transducer element) of a transducer device can be driven by electrical signals transmitted via a dedicated electrical link. Yet, it has been observed that an electrical noise (which may comprise in particular electronic noise) might disturb the electrical signals circulating in these electrical links, in particular because of the asymmetry of these electrical links with respect to one another. This electrical noise (also called later on disturbing noise, or noise) degrades the exchanged signals and disturbs driving of the wave emitter and/or receiver device, which could in particular cause a degradation of the quality of the image in imaging applications (for example ultrasound imaging).

Indeed, the electrical links used to transmit the electrical signals may for example comprise electrical tracks formed on an electronic board and/or electrical cables (for example of the coaxial type). Yet, physical disparities generally exist between several electrical tracks or links, in particular in terms of length, dimension, electrical conductivity, impedance, etc. Although electrical tracks could be designed so as to be identical or symmetrical, in practice, the existence of such physical or structural differences is inherent to making of electrical tracks or links. It has been observed that these asymmetries between the electrical links could be one of the origin of the disturbing noise.

This noise problem is further amplified by the increasing number of electrical links nowadays provided for in drive systems (in particular at the level of the electronic boards), which increases the constraints on routing or arrangement of such electrical links, for example in the electronic boards of the drive systems. Thus, it is increasingly difficult to adapt the design of electrical tracks on a drive board because of the design constraints (limited size of the board, increasing number of electrical tracks, increasing complexity of the board, etc.).

In particular, the number of electrical links necessary to drive a piezoelectric transducer device depends on the number of piezoelectric elements present in the piezoelectric transducer device. The greater the number of piezoelectric elements, the greater the number of necessary electrical links will be. The development of applications requiring a large number of piezoelectric elements (for example matrix array probes, 3D probes, etc.) leads to amplifying the problems of asymmetries and arrangement constraints described before.

It has been demonstrated that the electrical noise results in particular from physical phenomena such as the track length differences (“trace length mismatch”), the impedance differences (“impedance mismatch”), the propagation time offsets (“propagation-time mismatch”), the parasitic phenomena of coupling between the electrical links (called “crosstalk”), etc.

Moreover, it has been noticed that the disparities in terms of characteristics or behaviour between the electronic components used in a drive system, in particular in an electronic board, to drive a wave emitter and/or receiver device may also be at the origin of electrical noise(s) and therefore of degradation of the signals.

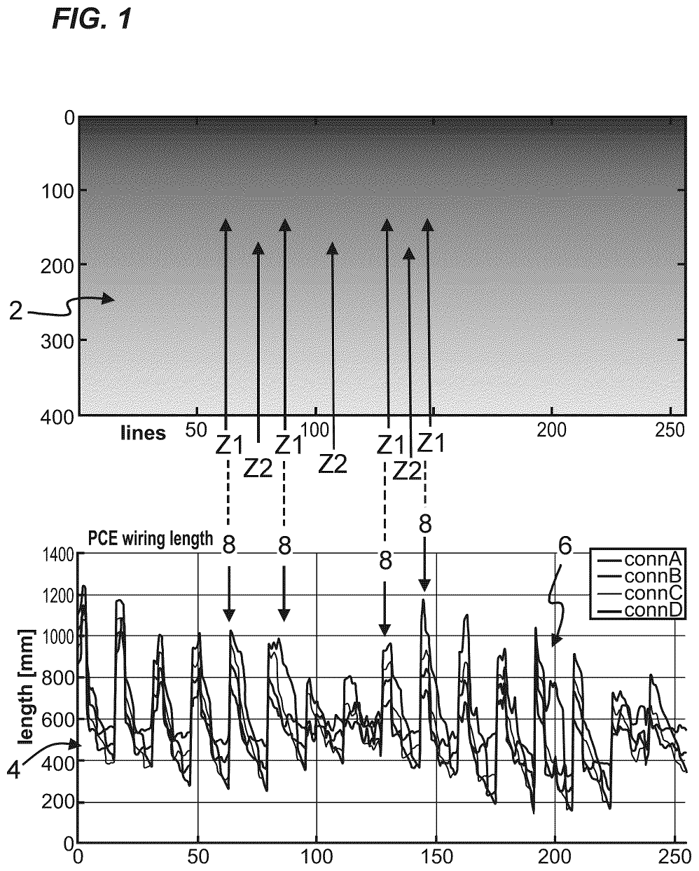

FIG. 1 illustrates according to a particular example an ultrasonic image 2 defined by grayscale levels which depend on the response of a medium to ultrasonic waves. In this image, the abscissa axis represents the position (or index) of the used piezoelectric elements and the ordinate axis represents the depth in the medium. As example, it is herein assumed that an ultrasound transducer device (not shown) is used comprising 256 transducer elements to probe the considered medium.

It has been noticed that a degradation of the quality of the ultrasonic image 2 by the effect of an electrical noise present at the electrical links between a drive system (not shown) and the ultrasound transducer device. This degradation which leads to a poorer and even very deteriorated image quality is reflected in particular by an alternation of dark areas Z1 and clear areas Z2 which is in no way representative of the studied medium. The dark areas Z1 of the image 2 correspond to channels that receive a very low-level signal. The clear areas Z2 are clearer areas (grey or white) corresponding, in the image 2, to signals with a higher level, but which are disturbed by measurement noise which is reflected by visual noise (and not real elements of the observed medium).

FIG. 1 also illustrates a graph 4 including four curves 6 each illustrating, for a given probe, the length of the electrical tracks (on the electronic board) used respectively to drive the piezoelectric elements of the transducer device. Thus, the abscissas of the graph 4 represents the position (or index) of the used piezoelectric elements whereas the ordinates represent the lengths of the electrical tracks (in millimetres in this example).

FIG. 1 shows the effect of the length disparities between the electrical tracks on the level of the electrical signals received by the piezoelectric elements of the probe. As illustrated, these curves 6 form a series of peaks 8 corresponding to electrical noise level maxima generated in the electrical links. As show in the curves 6, the level of the electrical noise varies depending on the used electrical links, each attenuation peak 8 corresponding in this example to a dark area Z1 in the image 2. The longer the used electrical track, the higher the electrical attenuation (or loss) will be, and therefore the weaker the received electrical signal will be (and therefore the darker the corresponding region of the image 2 will be).

Correction algorithms may be used to compensate for the image degradations resulting from such an electrical noise but these algorithms are not always effective enough, they do not always provide satisfactory results and complicate the processing of the signals and therefore increase the processing costs in terms of time and resource, which is barely compatible in particular with a real-time use of the system.

A method and a device for driving a wave emitter and/or receiver device will now be described in the following with reference to FIGS. 1-12 provided for illustration. Unless stated otherwise, elements that are common or similar in several figures bear the same reference signs and have identical or similar characteristics, so that these common elements are not generally described again for simplicity.

The terms “first” (or “first”, “second”, etc.) are used in this document by an arbitrary convention to allow identifying and distinguishing different elements (such as operations, devices, etc.) implemented in the embodiments described hereinafter.

As indicated before, the invention relates in particular to a method for controlling a wave emitter and/or receiver device. FIG. 1 schematically shows a system (or device) 10, also called control system or control device, configured to drive a wave emitter and/or receiver device 20 according to some embodiments of the present disclosure.

The device 20 is configured to emit and/or receive waves W. The nature of these waves depends on the configuration of the device 20, in particular with regards to the intended use thereof. According to one example, the device 20 is configured to emit waves W. According to another example, the device 20 is configured to receive waves W. According to another example, the device 20 is configured to both emit and receive waves W.

For example, the waves W may be (or comprise) acoustic waves, for example of the ultrasonic type. As example, it is considered later on that the device 20 is an ultrasonic transducer device (or ultrasound probe) which allows emitting and/or receiving ultrasonic waves. Nonetheless, it should be noted that other examples of wave emitter and/or receiver devices are possible according to the present disclosure as specified later on.

In this example, the control system 10 may comprise a control unit (or device) 12 (also called power control unit) configured to drive the transducer device 20 by means of electrical signals SG which are exchanged (or transmitted) between the system 10 and the transducer device 20.

More particularly, the control unit 12 is configured to generate electrical signals SG which are transmitted to the transducer device 20 to cause the emission, by said transducer device 20, of ultrasonic waves W1 in the direction and/or in a medium M. The electrical signals SG thus generated are representative of he (or define the) ultrasonic waves W1 projected in the medium M. To this end, the control unit 12 may, for example, be or comprise at least one electronic pulser or simply “pulser” (i.e. an electrical signal generator). The electrical signals SG may have various forms, for example a square form, a sine form, a random form, etc.

For example, the control unit 12 may comprise receiver devices or receiver circuits (not shown) configured to receive electrical signals SG originating from the transducer device 20.

As illustrated in FIG. 1, the system 10 may also comprise in this example a processing unit (or device) 11 configured to control the control unit 10, for example by controlling the electrical signals SG. For example, this processing unit 11 may be or comprise at least one processor.

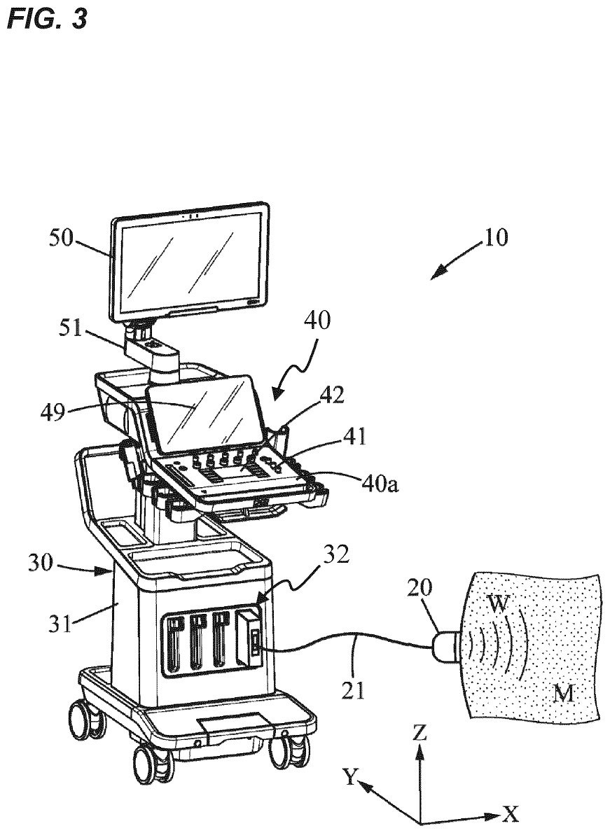

According to one example, the processing unit 11 and/or the control unit 12 are comprised within the body 31 (central element) of the system 10 shown in FIG. 3.

More particularly, the processing unit 11 may be configured to control the electrical signals SG which are generated by the control unit 12. The processing unit 11 may also be configured to process (or interpret) electrical signals SG received by the control unit 12 originating from the transducer device 20. These signals SG are representative of waves W2 received by the transducer device 20 originating from the medium M. For example, these waves W2 form one or more ultrasonic echo(es), i.e. a response of the medium M to the ultrasonic waves W1. The processing carried out by the processing unit 11 on the received signals SG may vary as the case may be and comprise for example at least any one of amplification, filtering, digitisation and conditioning operations of the signals SG.

The system 10 may comprise the transducer device 20. Alternatively, the transducer device 20 may be external to the system 10. For example, the transducer device 20 may be connected to the system 10 by a cable or may communicate wirelessly with it. In the last case, the transducer device 10 may for example comprise a battery and receive communication signals from the system 10 which represent the electrical signals SG (for example the drive frequencies and/or any information contained in the electrical signals). The transducer device 20 may then internally replicate the electrical signals SG from the received communication signals.

For example, the transducer device 20 may be a conventional wave emitter and/or receiver device. Thus, one difference according to the present disclosure may lie in the manner in which the transducer device 20 is driven and the associated means that are implemented to carry out such drive.

The system 10 may be stationary or mobile. The transducer device 20 may also be stationary or mobile. For example, the system 10 may be a fixed system (for example comprising a processing unit and a display device, as described hereinbelow) and the transducer device 20 may be mobile (for example a sensor device, a measuring device, or a probe). Nonetheless, the transducer device 20 may also be integrated into the system 10, and the system 10 may be a mobile system. For example, the system 10 may be configured to be driven in a standalone manner, for example thanks to an included battery. Other examples are described below.

As illustrated in FIG. 1, the system 10 further comprises a drive device (or drive unit) 60 configured to drive the transducer device 20. The configuration and the operation of the drive device 60 will appear in more detail in the embodiments described hereinafter.

According to one example, the system 10 may comprise at least one memory (not shown) used by the processing unit 11 to control the control unit 10. This memory may possibly belong to the processing unit 11. In some examples, the processor and the memory of the processing unit 11 may be incorporated into the system 10 illustrated in FIG. 3 or may be incorporated into a computer or a computer device connected in a communicating manner with the latter. The memory may store instructions to execute the (or at least one of the) methods described in the present document. In particular, this memory may store instructions for processing ultrasonic data and/or instructions for building illustrative images of the observed medium.

Depending on the configuration and the type of the considered computer device, the memory of the processing unit 11 may be volatile (such as the RAM), non-volatile (such as the ROM, flash, EEPROM, etc., memory or any other computer-readable storage device and/or medium as described hereinafter) or a combination of both. For example, the memory used by the processing unit 11 may comprise all or part of a graphic card memory (or video card), this memory type being in particular able to process and/or send image data that can be used to display one or more image(s) on a display screen (or unit).

The system 10 (in particular the processing unit 11) may comprise storage devices (removable and/or non-removable), including, yet without limitation, magnetic or optical disks or tapes (possibly belonging to the processing unit 11).

Furthermore, the system 10 (in particular the processing unit 11) may include one or more input device(s) such as a keyboard, a mouse, a pencil, a voice input, etc., and/or one or more output device(s) such as one or more screen(s), loudspeakers, a printer, etc. The environment may also comprise one or more communication connection(s), such as LAN, WAN, point-to-point, etc., connections. In some embodiments, the connections may be used to establish point-to-point communications, wired communications, wireless communications, etc.

The system 10 (in particular the processing unit 11) may further comprise at least some kind of a computer-readable medium. The computer-readable media may consist of any available medium that could be accessed by the processing unit 11 (in particular its processor(s)) or other devices comprising the operating environment. By way of example, and without limitation, the computer-readable media may comprise computer storage media and communication media. The computer storage media comprise removable and non-removable, volatile and non-volatile media, implemented in any information storage technology or method such as computer-readable instructions, data structures, program modules or other data. The computer storage media do not comprise the communication media.

The communication media integrate computer-readable instructions, data structures, program modules or other data in a modulated data signal such as a carrier wave or another carrier mechanism, and comprise any medium providing information. The expression “modulated data signal” designates a signal of which one or more features are fixed or modified so as to code information in the signal.

By way of example, and without limitation, the communication media comprise the wired media such as a wired network or a direct wired connection, and the wireless media such as the acoustic, RF, infrared, microwave media and other wireless media. The combinations of any one of the elements above must also be included in the field of application of the computer-readable media.

The system 10 may be a single computer operating in a network environment using logical connections with one or more remote computers. The remote computer may be a viewing station, a personal computer, a server, a router, a network PC, an approved device or another shared network node, and generally comprises a plurality or all of the elements described above as well as others not mentioned. The logical connections may include any method supported by the communication media available.

The system 10 may be an imaging system, for example in the medical field. The images generated by the system may be either analysed in real-time, for example by a user, or analysed later on and/or in a location other than that in which the system 10 is located.

The system 10 may be a medical system, for example an ultrasound system. Consequently, the device 20 may be a medical and/or ultrasound probe.

For example, the system 10 may be associated with an ultrasound probe 20, in order to study the medium M, in particular to gather ultrasonic data from such a medium M. The medium M thus observed may be of any kind as the case may be. For example, it may consist of living tissues and/or in particular of human tissues. The observation of a medium M comprising one or more mineral structure(s) for example is also possible (gravel, volcano, etc.).

For example, the ultrasound probe 20 may comprise one or more transducer element(s) (not shown in FIG. 1), each being configured to convert an electrical signal SG received from the system 10 into ultrasonic waves and/or vice versa. Thus, the transducer elements may be configured to transmit (a) waves (or pulses) W1 in the medium M and/or to receive (b) a plurality of ultrasonic signals W2 from the medium M, possibly in response to the transmission (a) of the waves W1. An array of transducers comprising a plurality of transducer elements may be used. The same transducer element(s) may be used to emit waves (or pulses) and receive the waves forming the response of the medium, or different transducer elements are used for the emission and reception of the waves. There may be one or more transmitting transducer elements and a plurality of receiving transducer elements. In another variant, a single transducer element may be used. The transducer elements may comprise piezoelectric crystals and/or other components that may be configured to generate and/or record and/or receive signals. For simplicity of the disclosure, it is considered in the following embodiments that the transducer elements are piezoelectric elements.

Various arrangements of the transducer element(s) are possible. For example, an array of transducers comprising a plurality of transducer elements may be used. For example, it is possible to provide for a linear array comprising a plurality (for example between 100 and 300) of transducer elements juxtaposed along an axis X (horizontal direction or direction of the array X). In one example, the array is adapted to carry out a two-dimensional (2D) imaging of the medium M, but the array could also be a two-dimensional array adapted to carry out a 3D imaging of the medium M. Consequently, an array of transducers may be used. However, the system may also comprise one single row of movable transducers in a probe, so that the 3D imaging could be carried out. The array of transducers may also include one or more row(s) of emission transduction elements and one or more row(s) of reception transduction elements. The array of transducers may also be a convex array comprising a plurality of transducer elements aligned along a curved row (for example in a curved probe). The same transducer element(s) may be used to transmit a pulse and receive the response, or different transducer elements are used for the transmission and the reception. There may be one or more transmitting transducer elements and a plurality of receiving transducer elements.

The ultrasound probe 20 may further comprise or be associated with an electronic control device (not shown in FIG. 1) controlling the transducer elements and acquiring signals SG from these. The electronic control device may be part of the probe. As a variant, the electronic control device may be external to the probe (for example, form part of the power control unit 12), or consist of a plurality of devices that partially form part of the probe and are partially external thereto.

Furthermore, the processing unit 11 may be configured to receive data from the transducer device 20, process data and/or send data to an external device, like for example, a processing, storage, display device, a server, a computer on which an artificial intelligence (IA) algorithm is executed, a dedicated workstation, or any other external device.

Nonetheless, the system 10 may be any type of electronic system. For example, the system 10 may be any type of medical system other than an ultrasound imaging system. Consequently, the transducer device 20 may be any type of imaging device or sensor, using waves other than the ultrasound waves (for example, waves having a wavelength different from the wavelength of the ultrasounds and/or waves not being sound waves).

According to other examples, the system 10 and/or the transducer device 20 are configured for communication, imaging or scanning purposes, for example in the medical imaging, radar, sonar, seismology, wireless communications, radio astronomy, acoustics and biomedicine fields.

Examples of medical imaging system comprise an ultrasound imaging system, an X-ray imaging system (for example a system allowing performing mammograms) and an MRI (magnetic resonance imaging) system.

FIG. 3 schematically shows embodiments of the system (or device) 10 as described before with reference to FIG. 1, namely an ultrasound imaging system in this example.

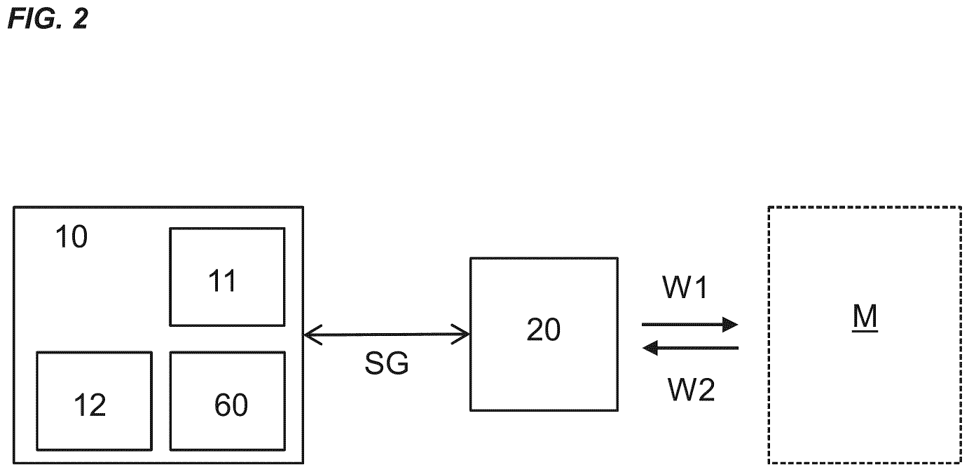

The ultrasound imaging system 10 as shown in FIG. 2 may comprise:
an ultrasound probe 20 (corresponding for example to the transducer device 20 of FIG. 1),
a processing unit 30 configured to process or generate an image based on the signals SG received by the probe 20 (this unit 30 corresponding for example to the processing unit 11 of FIG. 1),
a control panel 40 associated, linked or connected to the processing unit 30, said control panel possibly comprising at least one of the buttons 41 and a touch pad 42, and
one or several screen(s) 50 configured to display the image(s) generated by the processing unit 30.

The probe 20 may be connected to the processing unit 30 by any suitable connection means such as a cable 21 or a wireless connection. The probe 20 is further capable of emitting ultrasonic waves W in a medium M and of receiving ultrasonic waves W from the medium M, said received ultrasonic waves being the consequence or the result of reflections of said emitted ultrasonic waves on diffusing particles inside said medium.

The probe 20 may be (or comprise) an array of transducers comprising a plurality of transducers (not shown), each converting an electrical signal SG into a vibration and vice versa. For example, a transducer (also called transducer element) is a piezoelectric element. For example, the array of transducers may comprise 128, 256 transducers or more. The array of transducers may be linear or curved or may be arranged on an external surface of the medium M so as to be coupled with the medium and vibrate and emit or receive ultrasonic waves W.

The processing unit 30 may comprise receiver devices (not shown), comprising (or being) for example receiver circuits, configured to process (for example amplify and/or filter) the signals SG received from the probe 20. For example, these receiver devices may comprise converters to transform the received signals into data representing the signal (for example analog-to-digital converters (ADC) configured to transform a voltage into a digital code). The obtained data may be processed in various manners; they may in particular be temporarily stored in a memory accessible to the processing unit or processed directly to compute intermediate processed data (beam-formed data). The processing unit 30 may use any known processing method to generate and/or process one or more image(s) or map(s) or data based on the signals received from the probe, such as the formation of beams. The processed data associated to an ultrasound image may be:
a B-mode image of the medium (B-mode image) generally displayed in grayscale allowing visualising the organs inside the medium, and/or
a so-called Doppler image, showing the movements of fluids in the observed medium, for example the velocity of the movement and/or the flow of the fluids in the medium (image often using colour codes), for example useful to visualise the blood vessels and their activities in the medium, or
an image showing a mechanical characteristic of the medium (elasticity), for example useful to identify areas with different hardness which might turn out to be tumours present inside the medium (for example shear wave elastography image data (ShearWave™ Elastography, SWE) as described hereinbelow).

The display screen 50 may be a screen for visualising the image processed by the processing unit 30. The display screen 50 may also display other information such as the scales used in the image, or configuration information for the processing or any information such as assist information or a contextual gestural assistance adapted or adaptable to the touch pad 42.

The display screen 50 may be articulated on a support arm 51 for a better positioning opposite the user. For example, the display screen may be a large-size (at least 20 inches) screen for a better visualisation of the images by the user. An additional screen (not shown) may be present to show images to the patient or for any other use (for example in a surgical operating room).

The control panel 40a forms all or part of a user interface that can be used by a user to interact (control, receive information, etc.) with the system 10.

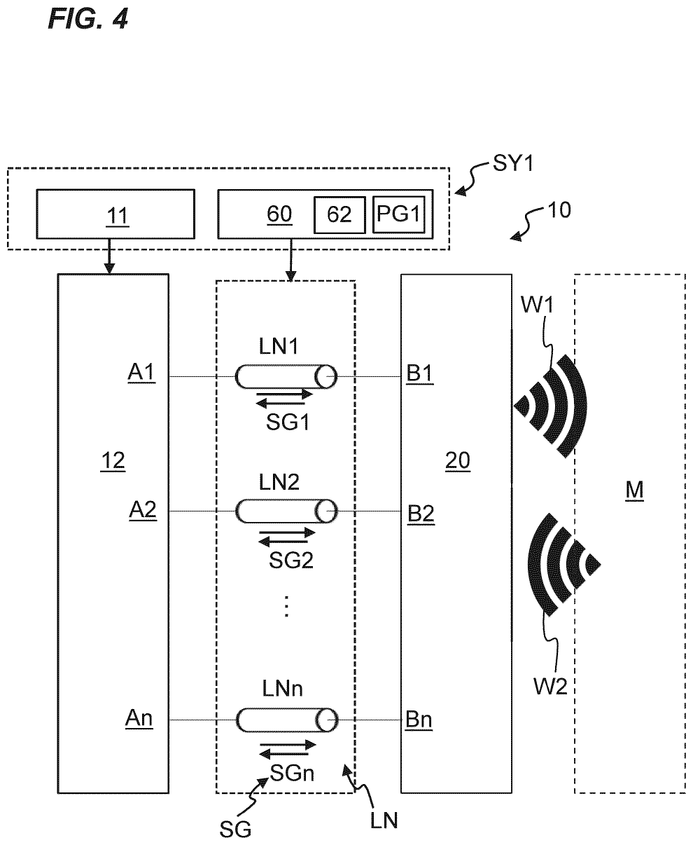

Examples of implementation of the system 10 according to the present disclosure will now be described hereinafter with reference to FIGS. 4-12.

FIG. 4 schematically shows embodiments of the system 10 as described before with reference to FIGS. 1 and 2, namely an ultrasound imaging system in this example.

As indicated before, the system 10 comprises in this example a control unit 12 configured to drive the transducer device 20 with electrical signals SG. Thus, the control unit 12 generates electrical signals SG which are sent to the transducer device 20 to cause the emission of ultrasonic waves W1 in the medium M. The electrical signals SG thus generated are representative of the ultrasonic waves W1 sent to excite the medium M. The control unit 12 is also able to receive electrical signals SG originating from the transducer device 20 following an excitation induced by the emitted waves W1. These signals SG are representative of waves W2 received by the transducer device 20 originating from the medium M. For example, these waves W2 form an ultrasonic echo, i.e. a response of the medium M to the ultrasonic waves W1.

The system 10 also comprises a processing unit (or device) 11 configured to control the control unit 12, in particular to control the electrical signals SG generated by the control unit 12. he processing unit 11 is further configured to process (or interpret) electrical signals SG received by the control unit 12 originating from the transducer device 20.

As illustrated in FIG. 4, the control unit 12 uses electrical links LN to exchange electrical signals SG with the transducer device 20, in emission and in reception. Next, it is considered that a plurality of electrical links denoted LN1 to LNn are formed between the control unit 12 and the transducer device 20, n being an integer greater than or equal to 2. This number n may be adapted as the case may be. For example, it is possible to set n such that n=256.

The electrical links LN may be (or comprise) electrically-conductive physical links (or connections) which are used to transport the electrical signals SG between the control unit 12 and the transducer device 20. The configuration of these links LN, in particular in terms of arrangement and structure, may vary as the case may be. For example, each link LN may be formed entirely or partially by at least one electrical track, for example one or more electrical track(s) formed over an electronic board (for example the drive board 32 illustrated in FIG. 3). Each link LN may possibly comprise at least one portion of a cable or an electrical cable (for example of the coaxial type), such as the cable 21 illustrated in FIG. 3. According to one example, such an electrical link LN may comprise at least one electrical track portion, and possibly also at least one electrical cable portion.

The electrical links LN may directly connect the control unit 12 to the transducer device 20 or form only one portion of the electrical connection connecting the control unit 12 and the transducer device 20.

As illustrated in FIG. 4, the control unit 12 comprises in this example n input/output terminals respectively denoted A1 to An and the transducer device 20 comprises n input/output terminals respectively denoted B1 to Bn. The electrical signals SG are then transmitted between the control unit 12 and the transducer device 20 via the terminals A1-An and B1-Bn connected together by the electrical links LN1 to LNn. As described hereinafter, the manner in which these electrical links LN are used to convey the electrical signals SG between the input/output terminals A1-An on the one hand and B1-Bn on the other hand may advantageously be adapted over time under the control of the drive device 60.

As example, it is assumed that each terminal B1 to Bn is associated to a respective piezoelectric element (not shown) of the transducer element 20. Thus, each piezoelectric element can be driven by means of electrical signals SG transmitted from an input/output terminal A1-An of the control unit 12. Nonetheless, variants are possible, in particular implementations wherein a terminal B1-Bn of the transducer device 20 is associated with a subgroup, or a group, or a plurality of piezoelectric elements.

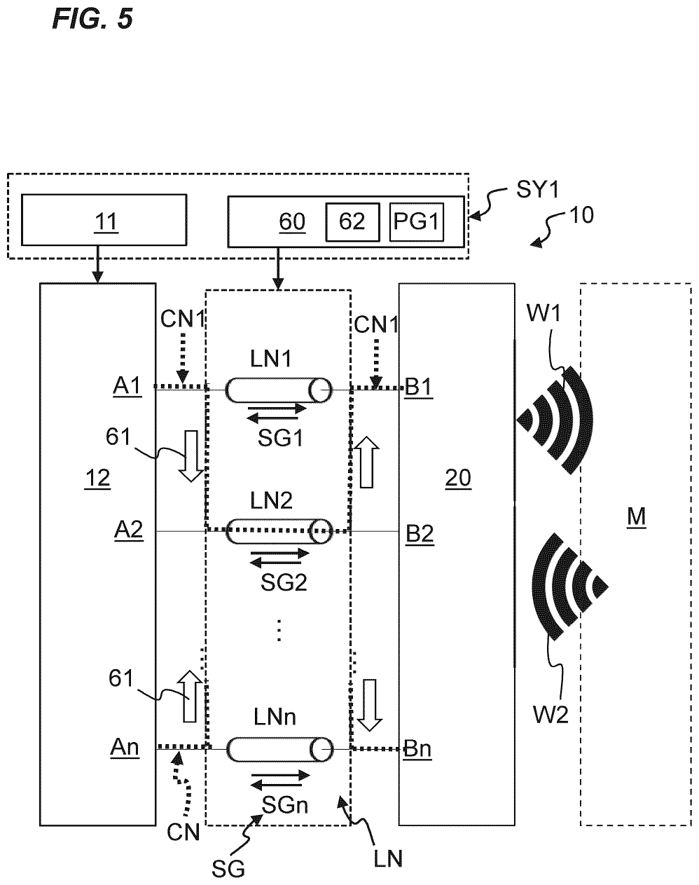

As illustrated in FIG. 5, the control unit 12 is configured to drive the transducer device 20 (also called first device) throughout channels CN1 to CNn (collectively denoted CN) established via the electrical links LN. Each channel CN forms an electrical route (or electrical pathway), among the electrical links LN, respectively connecting (or coupling) a terminal A1-An of the control unit 12 and a corresponding terminal B1-Bn of the transducer device 20.

According to one example, the channels CN are analog channels.

These enable the transmission and/or the reception of electrical signals SG between the control unit 12 and the transducer device 20 (transmission and/or reception channels). In other words, these channels CN are configured to convey the electrical signals SG between the control unit 12 and the transducer device 20.

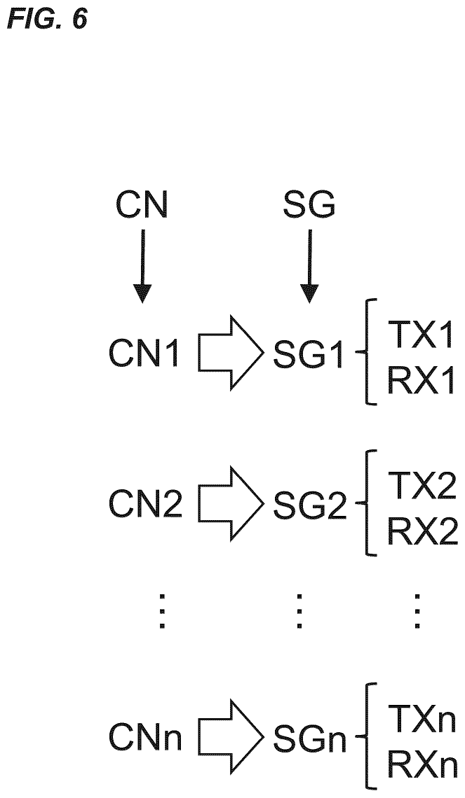

As illustrated for example in FIG. 6, each channel CNi (CN1 to CNn) may be configured to convey (or transmit) respectively electrical signals SGi (SG1 to SGn) between the processing unit 12 and the transducer device 20, i being an integer comprised between 1 and n. As indicated before, these exchanged signals SGi may comprise an electrical signal TXi emitted by the control unit 12 and/or an electrical signal RXi received by the control unit 12.

According to one example, the channels CN are wired channels (or galvanic connections) to the extent that they are formed by wired-type electrical links LN.

The drive device 60 is configured to carry out one or more channel CN permutation(s) 61 (FIG. 5) over time among the electrical links LN. Each permutation 61 results in a modification of the electrical pathway followed by at least two channels CN among the electrical links LN. All or only part of the channels CN may have their pathway modified during the same permutation.

To do so, the drive device 60 may comprise for example a drive module (or unit) configured to send permutation commands, each permutation command triggering a permutation 61 of at least two channels CN among the electrical links LN. In the present disclosure, such a module may correspond to both a software component, and a hardware component or to a set of hardware or software components.

As illustrated in FIG. 5, it is for example assumed that the channel CN1 coupling the terminal A1 of the processing unit 12 to the terminal B1 of the transducer device 20 is initially established via the electrical link LN1 and that, under the effect of a permutation 61 triggered by the drive device 60, said channel CN1 is reconfigured to be established via the electrical link LN2. Thus, the pathway of the electrical signals SG is modified between the terminals A1 and B1. In parallel, this permutation 61 may also cause another channel CN to be established via the electrical link LN1.

Next, for simplicity of the explanatory illustration, it is assumed that each channel CN is configured to connect together the same input/output terminal A1-An of the processing unit 12 to the same input/output terminal B1-Bn of the transducer device 20, and that being so independently of each performed permutation 61. More specifically, the channel CN1 couples the pair A1/B1, the channel CN2 couples the pair A2/B2, etc. at any time point over time. Thus, each channel permutation 61 results in a modification of the pathway of at least two channels CN among the electrical links LN without modifying the correspondences between the terminals A1-An of the processing unit 12 and the terminals B1-Bn of the transducer device 20. Hence, the permutations 61 are transparent from the perspective of the processing unit 12 and of the transducer device 20, which allows limiting the necessary adaptations at the level of the system 10 and not confusing the user and/or the designer of the system.

Various channel permutations 61 are possible in the context of the present disclosure. Examples of implementation of such permutations are described hereinafter.

According to one example, the channels CN may be configured so that only one electrical link LN is used at each time per channel CN. In other words, each of the channels CN is established in one single electrical link LN, and that being so independently of the performed permutation(s) 61. Nonetheless, variants are possible, for example implementations wherein the channels CN are established in several portions of different electrical links LN, as described later on.

According to one example, the channels CN may be configured so that at least one channel is used to drive an element PZ or a plurality of elements PZ (for example in the case of a matrix array probe).

As illustrated in FIG. 5, the drive device 60 may comprise at least one processor 62 and a non-volatile memory (not shown). The drive device 60, and more generally the control system 10, are configured so as to implement a drive method for driving the transducer device 20 as described hereinafter in particular embodiments. To this end, the drive device 60 may comprise a computer program PG1 stored in the non-volatile memory (Flash or ROM type memory for example), this computer program PG1 comprising all or part of the instructions for the implementation of said method. The processor 62 is configured to execute in particular the instructions defined by the computer program PG1.

The non-volatile memory 62 may correspond to any (removable and/or non-removable) storage device such as that one described hereinbefore with reference to the system 10. The drive device 60 may also comprise at least some kind of a computer-readable medium as described before with reference to the system 10 (for example computer storage media and/or communication media).

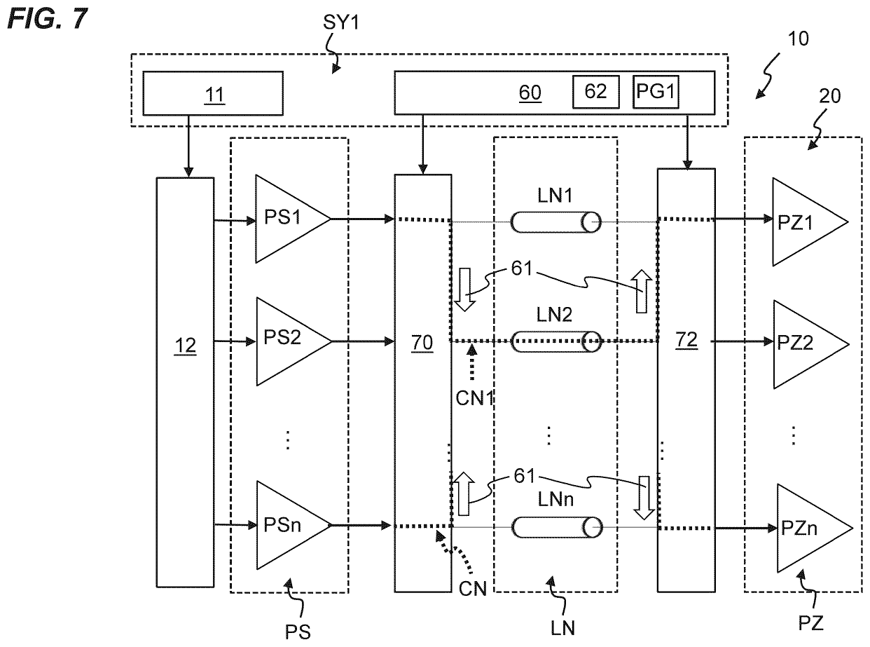

FIG. 7 schematically shows embodiments of the ultrasound imaging system 10 described before with reference to FIGS. 4-6. As illustrated, the system 10 comprises, for example, at least two multiplexers. Thus, the electrical links LN are interposed between a first multiplexer 70 and a second multiplexer 72. Each channel permutation 61 is be caused by a change in routing, by the first and second multiplexers 70 and 72 of the channels CN among the electrical links LN.

More specifically, the multiplexers 70 and 72 are configured to route the transmitted (or exchanged) signals SG between the control unit 12 and the transducer device 20 among the electrical links LN. This routing allows adapting the pathway of the signals SG among the electrical links, and therefore adapting the channels CN established via the electrical links LN.

The multiplexers 70 and 72 are driven by the drive device 60 to carry out one or more channel permutation(s) 61 over time. Each permutation 61 causes a change in routing of the multiplexers 70 and 72, causing a permutation 61 of at least two channels CN among the electrical links LN. According to one example, each permutation 61 causes at least two channels CN to be established respectively via an electrical link LN different from that one previously used just before said permutation.

For example, the multiplexers 70 and 72 may be of the semiconductor (or “solid state”) type which allows in particular a robust and dense mounting in an electronic board.

According to one example, the first multiplexer 70 is implemented in a hardware and/or software form. For example, the multiplexer 70 may be in the form of an integrated circuit, for example of the FPGA (“Field-programmable gate array”) type. The multiplexer 70 may also be implemented at least partially by means of a computer program, for example the program PG1 (FIGS. 4, 5 and 7).

According to one example, the second multiplexer 72 may be formed in a probe, such as an ultrasound probe 20 (FIG. 3), or in an electronic board, such as the drive board 32 (FIG. 3). This last variant may be advantageous in that it is less complex and still allows effectively limiting the effects induced by the electrical noise as explained hereinafter, to the extent that the asymmetries of electrical links LN could occur mainly in such a drive board.

As shown in FIG. 7, the system 10 may comprise a plurality of pulsers denoted PS1 to PSn (collectively denoted PS) controlled by the control unit 12 to transmit electrical signals SG to the transducer device 20. These electrical signals SG may be or comprise electronic pulses representative of waves W1 to be emitted in the medium M. The pulsers PS may belong to the control unit 12.

As already indicated, the transducer device 20 may also comprise a plurality of transducers (FIG. 7), namely piezoelectric elements denoted PZ1 to PZn (collectively denoted PZ). These piezoelectric elements PZ are configured to convert the electrical signals SG received from the pulsers PS into waves W1 emitted in the medium M, and to convert the waves W2 received from the medium M into electrical signals SG which are sent to the control unit 12 for processing by the processing unit 11. Thus, in this example, each channel CN1-CNn associates one pulser PS1-PSn with the same piezoelectric element PZ1-PZn, and that being so independently of the permutations 62 that could be triggered by the drive unit 60. Thus, each piezoelectric element PZ may be driven by a pulser PS via a dedicated channel CN.

According to one example, the system 10 (for example the control unit 12 or the processing unit 11) comprises receiver devices (not shown) as described hereinbefore in particular with reference to FIG. 3. These receiver devices, comprising (or being) for example receiver circuits, are configured to process (for example amplify and/or filter) signals SG received from the transducer device 20. For example, these receiver devices may comprise converters to transform the received signals into data representing the signal (for example analog-to-digital converters (ADC) configured to transform a voltage into a digital code). For example, each receiver device is configured to receive electrical signals SG from a piezoelectric element PZ throughout a respective channel CN.

As illustrated, the drive device 60 and the processing unit 11 may form together a system SY1, also called drive system. According to one example, the drive device 60 and the processing unit 11 form one (or belong to one) single device SY1.

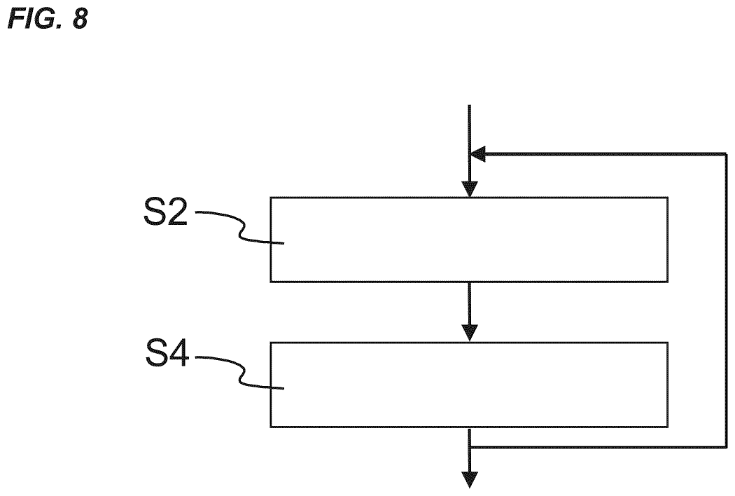

A control method implemented by the system 10 as described before (FIGS. 2-7) is now described with reference to FIGS. 8-11 according to particular embodiments. To do so, the drive device 60 (or more generally the system 10) may execute the computer program PG1. The system 10 may execute instructions of at least one computer program, including the program PG1.

During a drive step S2, the control unit 12 drives the transducer device 20 throughout channels CN established via the electrical links LN. The channels CN may be established according to various configurations as the case may be.

According to one example, driving S2 comprises establishing channels CN by the drive device 60.

According to one example, driving S2 comprises transmitting electrical signals SG throughout the channels CN. As indicated before, the channels CN may be used to transmit (or exchange) electrical signals SG between the control unit 12 and the transducer device 20. Thus, signals SG may be sent and/or received by the control unit 12 throughout the channels CN.

During a permutation step S4, at least two channels CN are permuted 61 over time among the electrical links LN. In the examples considered herein, the channel permutation(s) 61 is/are triggered by the drive device 60 controlling the multiplexers 70 and 72 (FIG. 7), for example by sending permutation commands to the multiplexers 70 and 72.

Thus, each permutation 61 results in a modification of the electrical pathway of at least two channels CN among the electrical links LN. All or only part of the channels CN may have their pathway modified during a permutation.

According to one example, at least one channel permutation 61 is carried out (S4) over time among the electrical links LN.

According to one example, a plurality of channel permutations 61 is carried out (S4) over time among the electrical links LN. Thus, it is possible to carry out a series of permutations 61 at different time points over time, these permutations modifying, each time, the configuration (or electrical pathway) of the channels CN among the electrical links LN.

Thus, by permuting at least two channels CN among the electrical links LN over time, it is advantageously possible to smooth or average the electrical noise likely to disturb the electrical signals SG transmitted between the control unit 12 and the transducer device 20, and thus limit the possible disturbances that might result from such an electrical noise. Indeed, the parasitic phenomena related in particular to asymmetries of the electrical links LN with respect to each other as well as to the other variations due to the manufacturing processes of the components and electronic boards, may be attenuated or limited by modifying the pathway of the channels CN among the electrical links over time. This results from an averaging effect (or smoothing, or redistribution effect over time and/or space) of the physical or structural disparities of the electrical links LN caused by the change in the pathway of the channels throughout said electrical links. In particular, the variations of the properties of the channels (impedances, etc.) between the channels may be reduced. Thus, it is advantageously possible to improve the performances of the system, in particular by improving the drive of the wave emitter and/or receiver device and/or by improving the received response originating from the wave emitter an/or receiver device, and that being so while offering some freedom in the design of the electrical links LN. Indeed, it is possible to form electrical links with different characteristics (for example with different lengths) and/or a large number of electrical links, the physical or structural disparities between these links being compensated at least partially by permutation of the channels CN.

By limiting the effects induced by the electrical noise, it is possible, for example, to improve the image quality, such as for example the quality of an ultrasonic image in an ultrasound imaging application. Thus, it is possible to avoid visual anomalies (for example a series of dark and clear areas) likely to be induced by the electrical noise, as explained hereinbefore in particular with reference to FIG. 1. Thanks to the method of the present disclosure, it is possible in particular to obtain a good image quality without it being necessary to execute an image correction algorithm, which allows limiting the processing costs, in particular in terms of time and resources, in particular in the cases of so-called real-time applications.

According to one example, the channels CN are permuted during the permutation step S4 so that one single electrical link LN is used at each time per channel CN. In other words, each channel CN is established in one single electrical link LN independently of the performed permutations. Thus, it becomes possible to vary the pathway of the signals SG throughout the various channels CN with a minimum complexity. Nonetheless, variants are possible, in particular wherein at least one channel CN is established via several link portion belonging to different electrical links LN as described hereinafter.

Next, it is assumed that a plurality of channel permutations 61 is carried out (S4) over time among the electrical links LN. The completion of a plurality of permutations allows guaranteeing a good averaging (or redistribution, or smoothing) effect of the electrical noise and therefore limiting the possible disturbances that might disturb the transmission of the electrical signals, which enables in particular a better drive of the transducer device 20 and/or a better image quality in the case of an imaging application.

As described hereinafter in various illustrative examples, values adapted to each case could allow achieving a trade-off on the frequency of the permutations 61 over time in order to optimise the averaging (or smoothing, or redistribution) effect of the electrical noise, to the extent that too frequent permutations over time could cause time losses, and possibly disturb to some extent the electrical signals SG conveyed throughout the channels CN.

According to one example, the permutations are configured according to a permutation frequency which is higher than the refresh rate of an image generated by the system 10, for example of an ultrasonic image in an ultrasound imaging application, so that the image displays an averaged value of the electrical noises. For example, the permutation frequency may be adjusted so as to obtain the best image quality as possible (a little imaging noise, with no disturbing visual effect).

According to one example, m channels CN are permuted during each permutation 61, m being an integer such that 2≤m≤n. This number m may be fixed or may possibly vary from one permutation to another. Thus, it is possible to trigger permutations in groups of m channels, i.e. by causing, at each permutation 61, a permutation of all of the n channels CN or only a subset (m<n) among these n channels CN (in this last case, at least one channel CN is not permuted).

Next, it is assumed that all of the n channels CN are permuted at each permutation 61 although variants are possible where only one subset (fixed and/or variable over time) of the channels N permutes during the permutations 61.

The permutations 61 during the permutation step S4 may be implemented in various manners, some embodiments of which are described hereinafter.

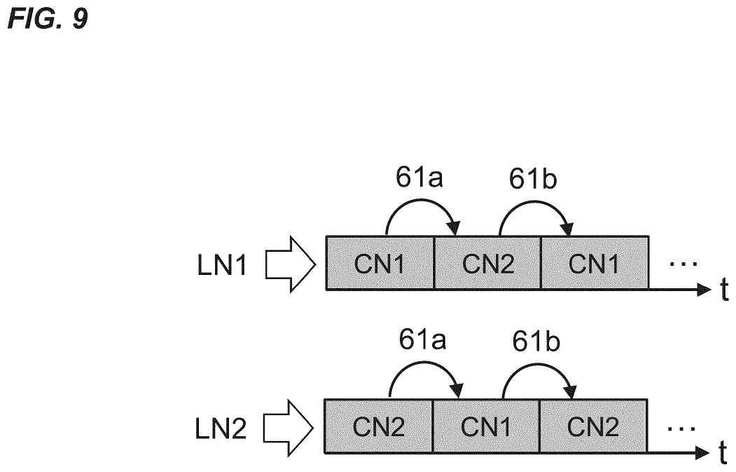

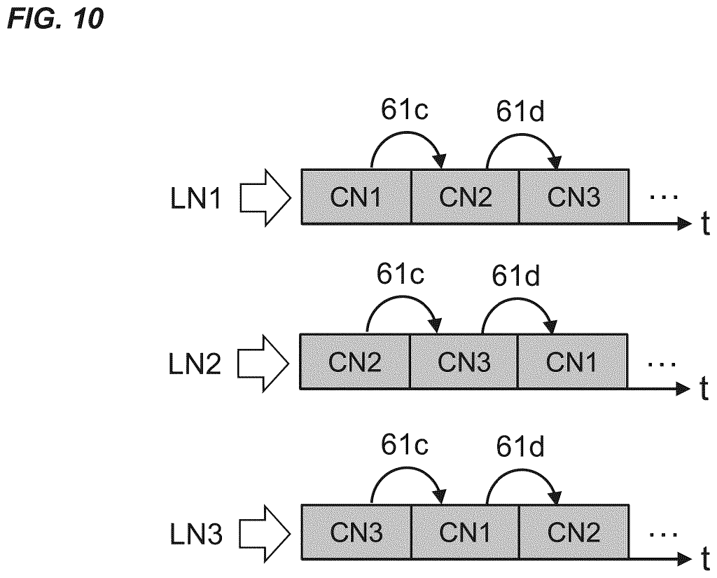

According to one example, the permutations 61 are carried out according to a predetermined sequence over time. For example, this predetermined sequence may define the electrical links LN in which the channels CN are respectively established upon completion of each permutation 61. Hence, in this case, the channel permutations 61 are defined in a deterministic way. The predetermined sequence may be independent of the electrical signals SG exchanged between the control unit 12 and the transducer device 20 (no servo-control of the channels CN according to the conveyed signals). FIGS. 9 and 10 described hereinafter illustrate drive examples based on deterministic permutations.

FIG. 9 is a time chart schematically illustrating the configuration of two channels CN1 and CN2 over time among the electrical links LN according to a simplified illustrative example. As illustrated in this example, two permutations 61a and 61b are successively carried out to permute together the channels CN1 and CN2. At an initial stage, it is considered that the channels CN1 and CN2 are respectively established via the electrical links LN1 and LN2. The first permutation 61a causes the channels CN1 and CN2 to be established respectively via the electrical links LN2 and LN1. The second permutation 61b causes the channels CN1 and CN2 to return back to their initial configuration by being established again respectively via the electrical links LN1 and LN2. This sequence may be repeated several times. Thus, the channels CN1 and CN2 interchange their electrical routes several times over time to achieve an advantageous effect of smoothing the variations of the induced disturbances as described before.

FIG. 10 illustrates a time chart schematically illustrating the configuration of three channels CN1, CN2 and CN3 over time among the electrical links LN according to one example. As illustrated in this example, two permutations 61c and 61d are successively carried out to permute together the channels CN1-CN3. In this example, at an initial stage, it is considered that the channels CN1, CN2 and CN3 are respectively established via the electrical links LN1, LN2 and LN3. The first permutation 61c causes the channels CN1, CN2 and CN3 to be established respectively via the electrical links LN3, LN1 and LN2 then the second permutation 61d causes these channels to be established respectively via the electrical links LN2, LN3 and LN1. Thus, the permutations may be repeated over time according to this same permutation scheme. Thus, the channels CN1, CN2 and CN3 are permuted over time so as to perform a “rotation”, or cyclic permutation, in the occupancy of the electrical links LN1, LN2 and LN3 allowing inducing an averaging effect as described before.

According to one example, the system 10 adapts the predetermined sequence of the permutations 61 according to at least one parameter, for example according to the used probe type (for example according to the frequency ranges used in the probes and/or according to the operating modes of the probes). For example, the predetermined sequence may be adapted according to one or more imaging parameter(s) implemented by the system 10, for example at least one of: a work frequency of the system 10, a number of piezoelectric elements PZ used in the system 10, a respective type of the piezoelectric elements PZ, an imaging mode implemented by the system 10 (such as the “B-mode” for example to generate grayscale images).

According to one example, the system 10 adapts the predetermined sequence of the permutations 61 according to an electrical noise present in the electrical links LN. Thus, the system 10 may, for example, analyse (or assess) an electrical noise (for example during an initial phase or calibration phase) disturbing the electrical links LN and determine the predetermined sequence according to the electrical noise thus analysed. The predetermined sequence of the permutations 61 can then be built from the electrical noise estimated during an initial or calibration phase.

The analysis of the electrical noise may be carried out in various manners. For example, this analysis may be done from an analysis of the images produced by the system 10 to determine whether these images are disturbed by electrical noise. For example, the analysis of the electrical noise may be carried out by means of an electrical measurement apparatus (comprising for example an oscilloscope) or by means of the system 10 itself. In this last case, the pulsers PS can then generate electrical signals and the receiver circuits of the system 10 can be used to measure the signals once transmitted throughout the electrical links LN, so as to be able to assess the attenuation of the signals and therefore the electrical noise affecting the electrical links LN.

According to one example, the permutations 61 are carried out according to a random (or pseudo-random) sequence over time. Thus, this random sequence may randomly define the electrical links LN via which the channels CN are respectively established upon completion of each permutation 61. In other words, the electrical link(s) LN via which each channel CN is established upon completion of a permutation may be randomly selected. The random nature of the permutations 61 allows further limiting the effects induced by the electrical noise and in particular improving the quality of an ultrasound image obtained from the signals SG processed by the processing unit 11. This is explained in particular by the particular operation of the human eye which generally perceives less the degradations of an image related to the electrical noise when the channels are randomly permuted (averaging effect on the human eye). In particular, it is possible to limit or avoid the periodicity of the defects (brightness variations, etc.) that might occur on an image so as to improve the visual rendering of the image.

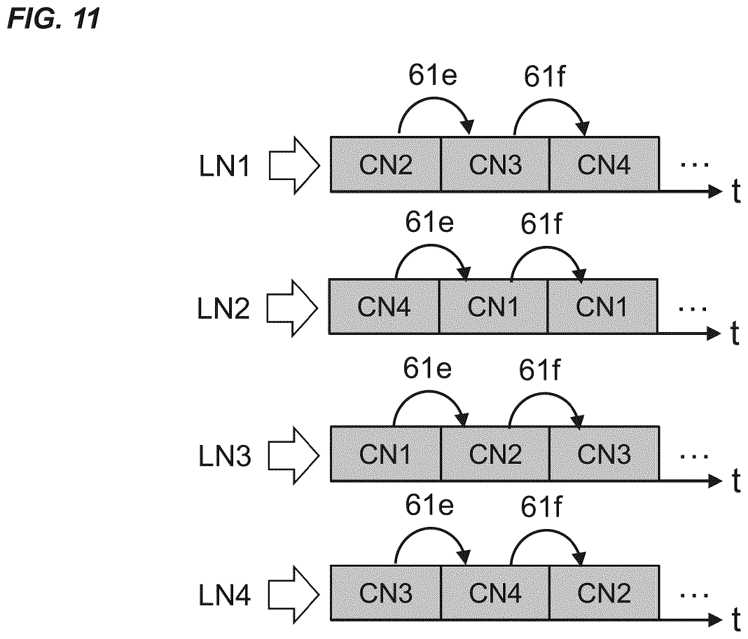

FIG. 11 illustrates an example of drive based on random permutations.

FIG. 11 illustrates a time chart schematically illustrating the configuration of four channels CN1-CN4 over time among the electrical links LN according to one example. As illustrated in this example, two permutations 61e and 61f are successively carried out to permute together the channels CN1-CN4. In this example, at an initial stage, it is considered that the channels CN1, CN2, CN3 and CN4 are respectively established via the electrical links LN3, LN1, LN4 and LN2. Each permutation 61e and 61f results in establishing the channels CN1-CN4 in an electrical link LN randomly selected among LN1-LN4. According to one example, at least one channel CN may possibly be kept in the same electrical link LN upon completion of a permutation whereas at least two other channels CN are permuted, although variants are possible where each channel CN necessarily changes electrical link LN at each permutation.

According to one example, the system 10 carries out permutations 61 according to a random combination of predetermined sequences.

According to one example, the system 10 carries out permutations 61 according to at least one sequence selected by a user among at least one candidate sequence, for example among at least one predetermined sequence generated by the system 10 and a random sequence. For example, the system 10 may determine candidate sequences and present to the user at least one image (or visual rendering) generated respectively from each candidate sequence, the user then being able to select at least one sequence of the candidate sequences using the images presented by the system 10.

As described before, the electrical links LN may be interposed between a first multiplexer 70 and a second multiplexer 72 (FIG. 7). Each permutation 61 may then be caused by a change in routing, by the first and second multiplexers 70 and 72 of the channels CN among the electrical links LN. These changes in routing may be triggered by the drive device 60 which controls the multiplexers 70 and 72. The multiplexers 70 and 72 may be arranged in the system 10 so as to cover/encompass electrical link sections that are the most likely to feature physical or structural disparities and therefore generate electrical noise.

The multiplexers 70 and 72 may be configured in various manners. According to one example, each channel CN may be configured to always connect the same pair of input/output terminals, namely a terminal A1-An on the one hand and a respective terminal B1-Bn on the other hand (FIGS. 4-5), and that being so independently of the performed permutation(s) 61. Furthermore, each input/output terminal A1-An of the control unit 12 may be associated with a respective pulser PS1-PSn and each input/output terminal B1-Bn of the transducer device 20 may be associated with a respective piezoelectric element PZ1-PZn. Nonetheless, other implementations are possible as described hereinafter.

According to one example, the multiplexers 70 and 72 are configured to carry out at least one among:
one (or at least one) first multiplexing of the channels CN between the plurality of pulsers PS and the plurality of piezoelectric elements PZ; and
one (or at least one) second multiplexing of the channels CN between the plurality of piezoelectric elements PZ and the plurality of receiver devices. Thus, the multiplexers 70 and 72 can carry out one or more first multiplexing(s) and/or one or more second multiplexing(s) as defined hereinbefore to permute the channels CN used respectively to emit and/or receive the electrical signals SG towards/from the transducer device 20.

According to one example, the multiplexers 70 and 72 carry out at least one first multiplexing of the channels CN between the pulsers PS and the piezoelectric elements PZ as described hereinbefore. According to a first variant, each channel CN (used to emit electrical signals SG towards the transducer device 20) then couples the same pulser PS to the same piezoelectric element PZ independently of the first multiplexing (and therefore independently of the permutations 61). Thus, each pulser may be coupled to the same unique piezoelectric element, irrespective of the permutations that are made. In this manner, the permutations 61 may be transparent for the control unit 12 and the transducer device 20. Thus, it is possible to implement the method while limiting the adaptations of the control unit 12 and of the transducer device 20 and while avoiding possibly confusing the user and/or the designer of the machine.

According to a second variant, the first multiplexing causes a change in coupling by the channels CN (used to emit electrical signals SG towards the transducer device 20) between the pulsers PS and the piezoelectric elements PZ. Thus, such a first multiplexing may lead a piezoelectric element PZ to be coupled with a different receiver device, before and after said first multiplexing. Thus, it is possible to permute the pulsers PS at the same time as the channels CN, i.e. modify the pulsers PS and the electrical links LN that are used to drive each piezoelectric element PZ.

According to a particular example, a piezoelectric element PZ may comprise a plurality of piezoelectric sub-elements PZ.

According to one example, the multiplexers 70 and 72 carry out at least one second multiplexing of the channels CN between the pulsers PS and the receiver devices as described hereinbefore. According to a first variant, each channel CN (used to receive electrical signals SG from the transducer device 20) couples the same piezoelectric element PZ to the same receiver device independently of the second multiplexing (and therefore independently of the permutations 61). Thus, each pulser PS may be coupled to the same unique receiver device, irrespective of the permutations that are made. In this manner, the permutations 61 may be transparent for the control unit 12 and the transducer device 20. Thus, it is possible to implement the method while limiting the adaptations of the control unit 12 and of the transducer device 20 and while avoiding possibly confusing the user and/or the designer of the machine.

According to a second variant, the second multiplexing causes a change in coupling by the channels CN (used to receive electrical signals SG from the transducer device 20) between the piezoelectric elements PZ and the receiver devices. Thus, it is possible to permute the receiver devices at the same time as the channels CN, i.e. modify the receiver devices and the electrical links LN that are used to receive the electrical signals SG from the piezoelectric elements PZ. According to a particular example, during the control method, the control unit 12 sends groups of electrical signals SG to the transducer device 20 throughout the channels CN, each group corresponding to a shot configured to cause the emission of one (or at least one) wave W1 by the transducer device 20. The channels CN (all or part of them) may be permuted for example every X shots among the electrical links, X being an integer greater than or equal to 2. Indeed, each channel permutation 61 is likely to generate electrical noise. To reach an adequate ratio between the reduction of the electrical noise resulting in particular from the line asymmetries and the secondary noise that might result from these permutations, it may be advantageous to limit the number of permutations over time, for example by triggering a permutation 61 only every X shots (where a shot corresponds for example to the emission of a wave W1). As example, the transducer device 20 may be driven by sending groups of 128 electrical signals (for example to generate a grayscale image) or of 1,000 electrical signals (for example to generate a colour image), each emission generating feedback signals allowing forming an image after processing. In this example, it is possible for example to set the number X to 2, 4 or 10.

Nonetheless, it is possible to set X such that X=1. In this case, the channels CN (all or part of them) may be permuted at each shot among the electrical links. More generally, it is therefore possible to set X such that X≥1.

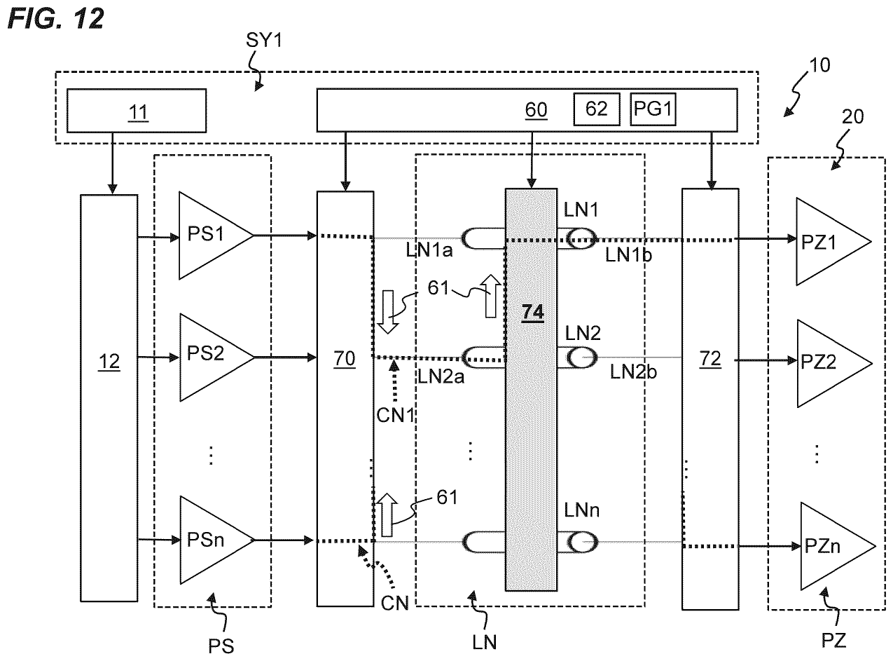

According to one example, the electrical links LN comprise at least one intermediate multiplexer dividing the electrical links LN into sections (or portions) mounted in series. In this case, the drive device 60 may carry out at least one permutation 61 which permutes the channels CN (all or part of the channels) among the sections of the electrical links LN. An embodiment of this example is now described with reference to FIG. 12, variants around this example being nonetheless possible.

FIG. 12 schematically shows the system 10 as described before, according to one embodiment. The system 10 differs from that one shown in FIG. 7 in that it further comprises at least one intermediate multiplexer 74. Next, for obvious reasons related to simplicity of explanation, it is assumed that the system 10 comprises one single intermediate multiplexer 74 although this variant could be applied similarly with a plurality of intermediate multiplexers 74.

More specifically, the intermediate multiplexer 74 “divides” (or separates) each of the electrical links LN into two sections (or portions) mounted in series. In other words, the addition of an intermediate multiplexing stage within the electrical links LN allows defining sections that can be driven individually, i.e. Sections among which independent permutations of the channels CN could be carried out. Thus, during the permutation step S4 (FIG. 8), the drive device 60 may for example carry out (or trigger) at least one permutation 61 which permutes the channels CN (all or part of the channels) among the sections of the electrical links LN. The permutation(s) 61 may be carried through a change in routing of the channels CN by the intermediate multiplexer 74 together with the multiplexers 70 and 72. In other words, besides the previously-described control of the multiplexers 70 and 72, the drive device 60 controls the intermediate multiplexer 74 so that the latter changes routing of the channels among the sections of the electrical links LN.

As example, it is assumed at an initial stage that the channel CN1 is established via a first portion LN1a of the electrical link LN1 and a second portion LN2b of the electrical link LN2. In response to the control of the drive device 60, the intermediate multiplexer 74 permutes (or modifies the routing) the channel CN1 so that it is established via a first portion LN2a of the electrical link LN2 and a second portion LN1b of the electrical link LN1. Thus, at least one channel CN may be established at a given time point via sections of at least two distinct electrical links LN.

Hence, the addition of at least one intermediate multiplexing stage within the electrical links LN allows refining driving of the transducer device 20 by enabling channel permutations 61 in sections, which increases the possible variations in routing of the channels and allows achieving an effective smoothing effect including with a limited number of electrical links LN. In particular, it is possible to primarily target the changes in routing at the level of the electrical line LN portions that are the most exposed to electrical noise, permanently or according to the use of the machine, i.e.; while varying over time and/or with the desired applications, in order to maximise the reduction of said electrical noise.

According to one example, the control method of the present disclosure, as described before in some embodiments, may be applied to ultrasound medical imaging. For example, the wave emitter and/or receiver device 20 is (or comprises) a medical probe intended to be used to study/scan (or examine) a medium M, for example all or part of a living being (a patient for example). To do so, the ultrasound probe 20 may, for example, be applied on the body of a living being, possibly while interposing a gel to improve the transmission of the ultrasound waves between the ultrasound probe 20 and the considered body.

According to one example, each electrical signal SG transmitted via the channels CN is configured to stimulate a piezoelectric element PZ (FIG. 7) of the medical probe 20 and/or represents an acoustic echo received by a piezoelectric element PZ of the medical probe 20.

As a person skilled in the art should understand, all of the above-described embodiments and variants some of which have been simplified in order to facilitate the explanations, are just but non-limiting examples of implementation of the present disclosure. In particular, a person skilled in the art could consider any adaptation or combination of the above-described embodiments and variants, in order to address a particular need.

Hence, the present invention is not limited to the above-described embodiments but covers in particular a control method that would include secondary steps without departing from the scope of the present invention. The same would apply to a drive device, or more generally to a drive system, for the implementation of such a method.

1. A method for driving a first wave emitter and/or receiver device, said method comprising:
driving the first device throughout channels established via electrical links; wherein at least two of said channels are permuted over time among the electrical links.

2. The method according to claim 1, wherein the driving comprises:
establishing the channels by a drive device.

3. The method according to claim 2, wherein the driving further comprises:
transmitting electrical signals throughout the channels.

4. The method according to claim 1, wherein said channels are analog channels for the transmission and/or reception of electrical signals between a control device and the first device.

5. The method according to claim 1, wherein the method comprises at least one permutation over time of said at least two channels among the electrical links.

6. The method according to claim 5, wherein the permutation comprises a plurality of permutations over time of the channels among the electrical links.

7. The method according to claim 5, wherein the channels are permuted during the permutation so that one single electrical link is used at each time per channel.

8. The method according to claim 5, wherein the permutation is carried out according to a predetermined sequence over time.

9. The method according to claim 5, wherein the permutation is carried out according to a random sequence over time.

10. The method according to claim 5, wherein the electrical links are interposed between a first multiplexer and a second multiplexer, each permutation being caused by a change in routing by the first and second multiplexers of the channels among the electrical links.

11. The method according to claim 10, wherein the first and second multiplexers carry out at least one of:
a first multiplexing of the channels between a plurality of pulsers and a plurality of piezoelectric elements; and
a second multiplexing of the channels between a plurality of piezoelectric elements and a plurality of receiver devices.

12. The method according to claim 11, wherein each channel couples the same pulser to the same piezoelectric element independently of the first multiplexing.

13. The method according to claim 11, wherein the first multiplexing causes a change in coupling by the channels between the plurality of pulsers and the plurality of piezoelectric elements.

14. The method according to claim 11, wherein each channel couples the same piezoelectric element to the same receiver device independently of the second multiplexing.

15. The method according to claim 11, wherein the second multiplexing causes a change in coupling by the channels between the plurality of piezoelectric elements and the plurality of receiver devices.

16. The method according to claim 10, wherein the electrical links comprise at least one intermediate multiplexer dividing said electrical links into sections mounted in series,
wherein the method comprises at least one said permutation which permutes the channels among the sections of the electrical links.

17. The method according to claim 1, wherein the driving comprises:
sending groups of electrical signals to the first device throughout the channels, each group corresponding to a shot configured to cause the emission of a wave by the first device; wherein said at least two channels are permuted every X shots among the electrical links, X being an integer greater than or equal to 2.

18. The method according to claim 1, wherein said waves are acoustic waves.

19. The method according to claim 1, wherein said method is applied to ultrasound medical imaging.

20. A computer program including instructions for the execution of the steps of a method according to claim 1 when said program is executed by a computer.

21. A computer-readable recording medium on which a computer program is recorded comprising instructions for the execution of the steps of a method according to claim 1.

22. A control system for driving a first wave emitter and/or receiver device, said system comprising:
a control device configured to drive the first device throughout channels established via electrical links; and
a drive device configured to permute at least two of said channels over time among the electrical links.

23. The system comprising the control system according to claim 22 and a second device driven by said control device.
